SEQUENCE LISTING (1 sequence)

SEQ ID NO: 1 - protein, 4 aa, Artificial Sequence
  Synthetic Peptide
KDVX

(19) United States
(12) Patent Application Publication
(10) Pub. No.: US 2014/0377390 A1
(43) Pub. Date: Dec. 25, 2014

(54) Cosmetic Compositions Comprising Plant Extracts for Combating Skin Aging

(71) Applicant: CASTER, Paris (FR)

(72) Inventors: Jean-Christophe Choulot, Rambouillet (FR); Neila Hajem, Croissy Sur Seine (FR)

(21) Appl. No.: 14/313,558

(22) Filed: Jun. 24, 2014

(30) Foreign Application Priority Data
Jun. 24, 2013 (FR) 1356013

(51) Int. Cl. A61K 8/97; A61Q 19/08; A61K 8/64

(52) U.S. Cl. CPC A61K 8/97; A61K 8/64; A61Q 19/08
USPC 424/775

(57) ABSTRACT

The subject of the present invention is a cosmetic composition comprising at least one silk tree extract, one pomegranate tree extract and one peptide of sequence SEQ ID NO: 1. The subject of the invention is also a cosmetic process for combating skin aging and/or withering, comprising the topical application of the composition to the skin.

The subject of the present invention is a cosmetic composition containing at least one silk tree extract, one pomegranate tree extract and one peptide of sequence SEQ ID NO: 1. The subject of the invention is also a cosmetic process for combating skin aging and/or withering, comprising the topical application of the composition to the skin.

Multidrug Resistance Proteins (MDRs) are transmembrane proteins present at the periphery of cells; these proteins act as “pumps” capable of excreting toxic substances out of the cell.

On cancer cells, the number of MDRs is particularly high. In these cells, the role of MDRs appears to be to allow said cells to reject chemotherapy drugs, perceived as toxic substances: MDRs allow cancer cells to resist drugs and prevent the elimination of said cells. This mechanism can thus explain why cancer cells are apparently immortal.

MDRs are also responsible for the resistance of bacteria to antibiotics.

On healthy cells, MDRs eliminate toxins of any nature, including molecules resulting from the glycation phenomenon, for instance Advanced Glycosylation End products (AGEs), via active expulsion of the endogenous toxic compounds out of the cell.

Longevity, and also the number of cell divisions, are linked to the amount and the functionality of MDRs, which lose most of their function at the end of reproductive life: these proteins therefore determine the lifetime of cells. A link has, moreover, been established between the MDR pathway and that of the SIRT1 protein, nicknamed longevity protein.

A study regarding MDRs has been carried out on yeasts, which represent a conventional model for studying aging (Eldakak et al., Nature Cell Biology, August 2010, vol 12 (8) 799-807). The particularity of yeasts is their proliferation by asymmetric division during which a “mother” cell will generate a “daughter” cell. During this division, the mother cell retains all the damaged proteins and other cell compounds that might be harmful, thus preserving the daughter cell. As a result, with aging, the cell accumulates intracellular toxins. During asymmetric division, MDR distribution occurs unequally:
the mother cell retains the aged original MDR proteins,
while the daughter receives the newly formed MDR proteins.
   The latter therefore has a better detoxification capacity.

If a yeast is mutated by suppression of the gene encoding an MDR, a reduction of from 11% to 66% in the number of daughter cells is then noted, which reflects a decrease in cell proliferation. If, on the contrary, yeasts are mutated and comprise an additional copy of each gene encoding the MDR protein, an increase of from 10% to 20% in the number of cell divisions is observed. In yeast cells in which division is asymmetric, the presence of MDRs in greater proportion makes it possible to increase their longevity. Thus, the link between cell longevity of a yeast colony and the role of MDRs is established.

In the mammalian organism, the only asymmetric division which takes place is that of stem cells. It is highly probable that, as for yeasts, an increase in MDR expression on stem cells will make it possible to increase their proliferative capacity, improving the longevity of the tissue concerned accordingly. It is particularly true for the epidermis. Indeed, contrary to the dermis, the epidermis contains a large number of stem cells located in the basal lamina.

During aging, the MDR proteins are less and less functional: the cells accumulate more and more intracellular toxins that it will be increasingly difficult to eliminate.

Thus, stimulating MDR activity makes it possible to preserve the ability of cells to eliminate endogenous toxic compounds, to improve cell detoxification capacity, to ensure cell regeneration and, as a result, to slow down the associated aging phenomena:

The applicant has developed novel cosmetic compositions, which constitute the subject of the invention.

A subject of the invention is also a cosmetic process for combating the effects of skin aging and/or withering, comprising the application to the skin of a composition as defined above.

Other subjects will emerge from reading the description and the examples which follow.

The compositions in accordance with the invention comprise, in a physiologically acceptable medium, at least one silk tree extract, one pomegranate tree extract and one peptide of sequence SEQ ID NO: 1.

The silk tree extract used comes from Albizia julibrissin. This tree, also called silk tree or mimosa, is a deciduous tree of the family Mimosaceae. It originates from Eastern and Southern Asia, but it has been spread over almost all the continents by man.

Albizia julibrissin extracts have already been proposed in the cosmetics industry. Document JP2009242296 describes an Albizia julibrissin extract capable of an inhibitory action on melanin production and a stimulatory action on collagen production. Document KR20100090530 describes the metalloproteinase-inhibiting activity of an Albizia julibrissin cortex extract. Document KR20020080657 describes a free-radical scavenging activity of an Albizia julibrissin bark extract. Document JP4342519 describes the use of an Albizia julibrissin extract for inhibiting tyrosinase activity. Document JP2000143488 describes the humectant properties for the skin and the hair of an Albizia julibrissin extract. Document JP2048515 describes the use of an Albizia julibrissin extract in a hair product for preventing hair loss and promoting the hair growth. This plant has demonstrated protective properties (glycation, lipofuscin, reinforcement of the activity of glyoxalase and of the proteasome, microvascular network protection and repair).

Preferably, the bark of this tree is used.

The method for obtaining this extract is preferably the following: the Albizia Julibrissin bark powder comes from PMA 28 or from the Herboristerie Caillaud. This Albizia julibrissin bark powder is subjected to a hot aqueous extraction under reflux. The extract obtained is then mixed with glycerin. Thus, the silk tree extract is preferably an aqueous-alcoholic extract.

In the compositions of the invention, the final concentration of dry silk tree extract is from 0.1% to 10% by weight of the total composition.

The pomegranate tree bears the Latin scientific name Punica granatum. It is a shrub 2 to 5 m high which belongs to the botanical family Punicaceae. It probably originates from Persia. The trunk, covered with a thin greyish bark, is unevenly branched into more or less spiny branches bearing shiny, dark green, whole oval opposing leaves with a short leafstalk and no stipule. The red, purple or garnet flowers are single at the axil of the leaves or combined by groups of 2 or 3 (G. Bezanger-Beauquesne L., Debraux G., Ressources Médicinales de la Flore Française [Medicinal Resources of French Flora], Vigot Frères Publishers, 1961, p. 838-842).

The parts of the pomegranate tree that are used are the root bark, the peel of the fruit, the flowers and the juice of the fruit. These various parts can be found described in the former Spanish, American and French pharmacopoeias. The pomegranate tree flower is preferably used. The pomegranate tree flower extract, containing antioxidant compounds, prevents UV-associated cytotoxicity by reducing MMP-1 synthesis, and thus reestablishing cell vitality after irradiation.

The extraction process is the following:

The dried pomegranate tree flowers from PMA 28 or from Herboristerie Caillaud are ground and then extracted in ethanol with stirring at ambient temperature under reflux.

Once the extraction has been carried out, the solution is filtered and then concentrated and dried in order to obtain a powder. The pomegranate tree flower extract is thus preferably an aqueous-alcoholic extract.

In the compositions of the invention, the final concentration of pomegranate tree flower extract is from 0.1% to 10% by weight of the total composition.

The peptide used is tripeptide-9 citrulline having the sequence H-Lys-Asp-Val-Cit-NH₂(SEQ ID NO: 1). It is a synthetic peptide obtained by reacting lysine and valine with 5-ureido-2-aminopentanoic acid (Lipotec, Spain). This peptide has the capacity to protect cell DNA from oxidation and from glycation.

Preferably, this peptide is used at a concentration of from 0.01% to 10% by weight of the total composition.

The composition according to the invention is suitable for topical application to the skin and therefore generally comprises a physiologically acceptable medium, i.e. which is compatible with the skin. It is preferably a cosmetically acceptable medium, i.e. a medium which has a pleasant colour, odour and feel and which does not cause unacceptable discomfort (tingling, tautness, redness), which may dissuade the consumer from using this composition.

The composition according to the invention may advantageously comprise an aqueous phase. The composition may comprise water in a content ranging from 1% to 99% by weight relative to the total weight of the composition, preferably ranging from 40% to 75% by weight relative to the total weight of the composition.

The composition according to the invention may be in any of the galenical forms normally used for topical application to the skin, in particular in the form of an optionally gellified oil-in-water or water-in-oil or multiple emulsion, a silicone emulsion, a microemulsion or nanoemulsion, a liquid, pasty or solid anhydrous product, or a dispersion of oil in an aqueous phase in the presence of spherules, it being possible for these spherules to be polymeric nanoparticles such as nanospheres and nanocapsules or, better still, lipid vesicles of ionic and/or non-ionic type.

This composition may be more or less fluid and may have the appearance of a white or coloured cream, an ointment, a milk, a lotion, a serum, a paste, a foam or a gel. It may optionally be applied to the skin in the form of an aerosol. It may also be in solid form, and for example in the form of a stick.

Advantageously, the composition according to the invention is in the form of an oil-in-water emulsion or of a gel, a cream or a stick.

In a known manner, the composition of the invention may also contain the adjuvants which are customary in the cosmetics and dermatological fields, such as hydrophilic or lipophilic gelling agents, hydrophilic or lipophilic agents, moisturizers (such as glycerin, propylene glycol, butylene glycol, pentylene glycol, hexylene glycol, dipropylene glycol or diethylene glycol), preservatives, antioxidants, solvents, fragrances, fillers, pigments, hydrophilic screening agents, odour absorbers and colorants. The amounts of these various adjuvants are those conventionally used in the fields under consideration, and for example from 0.01% to 20% of the total weight of the composition. These adjuvants, depending on their nature, can be introduced into the fatty phase, into the aqueous phase, into the lipid vesicles and/or into the nanoparticles.

When the composition according to the invention is an emulsion, the proportion of the fatty phase may range from 5% to 50% by weight, and preferably from 5% to 30% by weight, relative to the total weight of the composition.

As fats that can be used in the invention, mineral oils, oils of animal origin, synthetic oils, silicone oils and fluorooils may be used. Fatty acids, waxes and gums, and in particular silicone gums, may also be used as fats.

The emulsifiers and coemulsifiers optionally used in the composition in emulsion form are chosen from those conventionally used in the field under consideration. These emulsifiers and coemulsifiers are preferably present, in the composition, in a proportion ranging from 0.3% to 20% by weight, and preferably from 0.5% to 5% by weight, relative to the total weight of the composition. As emulsifiers and coemulsifiers that can be used in the invention, it is particularly advantageous to use fatty acid esters of a polyol, such as PEG-100 stearate, PEG-50 stearate and PEG-40 stearate, sorbitan tristearate, oxyethylenated sorbitan stearates available under the trade names Tween 20 or Tween 60, for example, and mixtures thereof.

As hydrophilic gelling agents, mention may in particular be made of carboxyvinyl polymers (carbomer), acrylic copolymers such as acrylate/alkyl acrylate copolymers, polyacrylamides, polysaccharides, natural gums, and clays, and as lipophilic gelling agents, mention may be made of modified clays such as bentones, metal salts of fatty acids, and hydrophobic silica.

Of course, those skilled in the art will take care to select the optional compound(s) to be added to the compositions according to the invention, and also the concentration thereof, in such a way that the advantageous properties intrinsically associated with the compositions in accordance with the invention are not, or not substantially, impaired by the envisaged addition.

The composition defined above is used by topical application in the context of a cosmetic process for combating skin aging and/or withering.

The invention will now be illustrated by means of the following non-limiting examples.

### EXAMPLE 1

### Evaluation of the Cytotoxicity of the Composition According to the Invention

This test serves to define the maximum tolerable dose that can be used on the cells without a toxic effect, by means of a rapid and sensitive quantification of the cell proliferation and of the cell viability as a function of the compositions according to the invention.

Briefly, the colorimetric test is based on the activity of a mitochondrial enzyme, succinate dehydrogenase, which degrades the yellow substrate MTT (3-[4,5-dimethylthiazol-2-yl]-2,5-diphenyltetrazolium bromide) into formazan crystals (violet). The amount of formazan salt produced by the cells from the MTT indicates the cell vitality and is measured by spectrophotometry at 570 nm.

The cells to be tested are primary human fibroblast cells (HDFa) (Tebu-Bio—Cat 106-05a; batch 2138—taken from facial plastic surgery on a 42-year-old Caucasian woman). In passage 3, the cells were seeded into 96-well plates in a proportion of 2000 cells in a volume of 200 μl of culture medium (Tebu-Bio—Fibroblast Growth Medium—500 ml—Ref 95116500).

These cells are then incubated for 24 h at 37° C. and 5% CO₂ so as to allow adhesion to the plate. On day 7 after seeding of the cells (the medium having been replaced with fresh medium every two days), the culture medium (Tebu-Bio—Fibroblast Growth Medium—500 ml—Ref. 95116500) is removed and 200 μl of the active agent to be tested, diluted in culture medium (no less than 50% of medium), are added and then the cells are reincubated (37° C., 5% CO₂).

The compositions are prepared in the following way:

For the silk tree extract, the concentrations used are 0.005%, 0.002%, 0.001%, 0.0008%, 0.0006%, 0.0004%, 0.0002%, 0.0001% and 0.00001%.

For the peptide of sequence SEQ ID NO: 1 used, the concentrations are 0.005%, 0.0002%, 0.0001%, 0.00008%, 0.00006%, 0.00004%, 0.00002%, 0.00001% and 0.000001%.

For the pomegranate flower extract, the concentrations used are 2%, 1%, 0.8%, 0.6%, 0.4%, 0.2%, 0.1%, 0.05% and 0.01%.

On the day of the test, 2 ml of fresh MTT solution are prepared by diluting the MTT powder (Thiazolyl Blue Tetrazolium Bromide, Sigma ref. M 5655) to 5 mg/ml in PBS.

20 μl of MTT solution are added to each well. After homogenization, the plate is reincubated (37° C., 5% CO₂) for 3 to 4 hours so as to allow metabolization of the MTT.

During this time, the MTT solubilization solution is prepared (DMSO/Isopropanol—1:1). The culture medium is removed by suctioning, and then the plate is dried with absorbent paper. The formazan crystals which have formed are then resuspended with 150 μl of MTT solubilization solution, by agitation at 150 rpm for 15 minutes at ambient temperature.

The optical density is then read at 570 nm on a Perkin Elmer Enspire® Multimode Reader spectrophotometer, with the OD at 670 nm (background noise) being removed.

The result is expressed as a function of the % absorption relative to the % absorption noted for the control cells (not treated with a compound).

The results for the various products tested are given in the table below (% cell vitality relative to the untreated cells, the vitality of which is 100%):

| | Silk tree extract concentration (as %) | | | | |
|---|---|---|---|---|---|
| | 0.005 | 0.002 | 0.001 | 0.0008 | 0.0006 |
| % vitality | 75.9 | 96.7 | 82.8 | 89.6 | 101.0 |

| | Silk tree extract concentration (as %) | | | |
|---|---|---|---|---|
| | 0.0004 | 0.0002 | 0.0001 | 0.00001 |
| % vitality | 107.7 | 92.0 | 102.9 | 101.1 |

For the peptide

For the peptide

| Peptide | 0.005 | 0.0002 | 0.0001 | 0.00008 | 0.00006 |
|---|---|---|---|---|---|
| concentration | | | | | |
| (as %) | | | | | |
| % vitality | 49.7 | 47.6 | 39.8 | 57.3 | 44.7 |
| Peptide | 0.00004 | 0.00002 | 0.00001 | 0.000001 | |
| concentration (as | | | | | |
| %) | | | | | |
| % vitality | 72.0 | 66.9 | 71.0 | 94.6 | |

For the pomegranate flower extract

| | Pomegranate flower extract concentration (as %) | | | | |
|---|---|---|---|---|---|
| | 2 | 1 | 0.8 | 0.6 | 0.4 |
| % vitality | 57.1 | 74.0 | 79.5 | 87.9 | 94.6 |

| | Pomegranate flower extract concentration (as %) | | | |
|---|---|---|---|---|
| | 0.2 | 0.1 | 0.05 | 0.01 |
| % vitality | 99.2 | 110.8 | 117.3 | 126.0 |

A final series of tests was carried out with the solutions S₀ to S₈ corresponding to successive dilutions of a solution S₀ containing 0.001% of silk tree extract, 4×10⁻⁵% of peptide and 0.2% of pomegranate tree flower extract.

| S₀ | S₁ | S₂ | S₃ | S₄ |
|---|---|---|---|---|
| | S₀/1.33 | S₀/1.5 | S₀/2 | S₀/5 |

| S₅ | S₆ | S₇ | S₈ |
|---|---|---|---|
| S₀/10 | S₀/20 | S₀/50 | S₀/100 |

Under these conditions, the results are the following.

| | Mixture solutions | | | | |
|---|---|---|---|---|---|
| | S₀ | S₁ | S₂ | S₃ | S₄ |
| % vitality | 79.9 | 81.2 | 88.2 | 85.3 | 88.2 |

| | Mixture solutions | | | |
|---|---|---|---|---|
| | S₅ | S₆ | S₇ | S₈ |
| % vitality | 58.2 | 98.5 | 96.5 | 109.5 |

According to these results, the concentration S2, which makes it possible to preserve close to 90% cell viability, is chosen for the rest of the tests. It corresponds to concentrations of:

| Silk tree extract | Peptide | Pomegranate flower extract |
|---|---|---|
| 6.6 × 10⁻⁴% | 2.6 × 10⁻⁵% | 0.13% |

### EXAMPLE 2

### MDR Activity Test

MDRs are transport proteins which make it possible to expel “undesirable” molecules out of the cell, thereby making it possible to detoxify the cell. By testing the MDR cell activity after treatment with active agents, it will be possible to deduce therefrom the capacity of the active agents to increase the cell detoxifying capacity.

In normal cells, non-fluorescent calcein enters the cell and accumulates therein. It is hydrolysed therein by endogenous esterases, and becomes fluorescent. In cells in which MDR expression is increased, the presence of these protein transporters makes it possible to expel the calcein out of the cell before it is cleaved: there is no fluorescence.

To do this, the Molecular Probes Vybrant MDR Resistance Assay Kit (V13180 Invitrogen Life Science) is used.

Thus, in this test, the weaker the fluorescence, the more active are the MDRs.

On DO, as many cell culture-treated, clear-bottom, black 96-well plates (Corning) as necessary are prepared with 7000 HDFa cells/well (human primary fibroblast cells (Tebu-Bio—Cat 106-05a; batch 2138—taken from facial plastic surgery on a 42-year-old Caucasian woman)) in a culture medium volume of 150 μl/well. These cells are then in passage 4. The cells are left to stand for 48 h for adhesion in an incubator at 37° C. and 5% CO₂. On D2, the culture medium is removed and is replaced with 150 μl/well of active agent diluted in culture medium, while providing five wells for each concentration of active agent, and in particular:
5 wells with PBS (which will not be treated with calcein, blank);
5 wells with PBS (which will be treated with calcein, control);
5 wells with verapamil at 30 μg/ml final concentration in the well (negative control. For this, 1 ml of absolute ethanol is added to the vial of 10 mg of verapamil (component C of the Invitrogen Life Science kit V13180) and vigorous agitation is carried out until the verapamil has completely dissolved. This stock solution at 10 mg/ml is then diluted in PBS until the working concentration is obtained;
5 wells with FGF (Sigma) at 5 nM final concentration in the well (positive control) or KGF (Sigma) at 25 ng/ml.

The cells comprising active agents are incubated at 37° C., 5% CO₂ for 24 hours.

On D3, a labelling solution is prepared by diluting the calcein solution of the kit at 1 mM (component A of the Invitrogen Life Science kit V13180) in PBS, so as to achieve a concentration of 1 μM (1000× dilution); 50 μl of calcein diluted to 1 μM are provided per well, i.e. 5 ml per 96-well plate. A lysis solution, which is water containing 0.25M of sucrose (Sigma), and 0.1M EDTA (Sigma), is then prepared.

To carry out the test, 50 ∥l of PBS are added to the control wells, and 50 ∥l of labelling solution are added to all the other wells. For each well, care is taken to homogenize the medium in the well.

The wells are left to incubate for 1 hour. The supernatent is then removed, and the cells are rinsed by adding 200 μl of culture medium (4° C.). This washing is repeated twice, and then the wells are emptied.

150 μl of lysis buffer solution are added to all the wells of the plate.

The calcein retention is measured on a Perkin Elmer Enspire® Multimode Reader fluorescence spectrophotometer, by reading the fluorescein fluorescence (λ_{excitation}: 494 nm, λₑₘᵢₛₛᵢₒₙ: 512 nm).

For each well, the percentage MDR activity for each of the active agents tested is calculated:
f₀ being the basal level, which is the value measured for the wells containing the control cells (calcein, but not treated with an active agent),
f_{Ax} being the ratio measured for each of the other wells (cells treated with the x active agents),
the percentage MDR activity is then: f_{AX}×100 /f₀.

The results (% calcein retention) are the following:

| | |
|---|---|
| Blank: no signal | 1.7% |
| Control (untreated cells) | 100.0% |
| 25 ng/ml KGF | 92.3% |
| 5 nM FGF | 91.8% |
| Verapamil at 30 μg/ml | 139.1% |
| Solution S₂ according to example 1 of the invention | 79.8% |
| Diamond Sirt at 0.015% | 87.5% |
| Hydrolysed soya protein fraction at 1% | 84.2% |

The Diamond Sirt product is distributed by Akott France of the Infinitec laboratories, and is composed of Ti dioxide microspheres covered with diamond aggregates and grafted with a biomimetic peptide of Sirt-1.

The hydrolysed soya protein fraction is sold by the company Ashland under the name Dynachondrine.

If the percentage obtained is less than 100, the active agent stimulates MDR activity. In the case where the percentage obtained is greater than 100, the active agent inhibits MDR activity.

It is thus possible to inhibit the signal, which reflects an improvement in MDR protein activity, with growth factors (KGF or FGF), but especially with:
The solution of example 1, a mixture of silk tree extract, of DNA-protecting peptide and of pomegranate flower extract;
A biomimetic peptide of sirtuin;
A soya extract fraction acting on mitochondria which has a multiple action, in particular on sirtuin-3, on the respiratory chain, on free radicals, on the ATP synthesis, etc.).

### EXAMPLE 3

### Day Cream

| Phase | Ingredients | % |
|---|---|---|
| A | Osmosed water | QS 100 |
| A | Glycerin | 3 |
| A | Preservative | QS |
| B | Acrylates/C10-30 alkyl | 0.5 |
| | acrylate crosspolymer | |
| C | Xanthan gum | 0.1 |
| D | Cetearyl olivate and | 2 |
| | sorbitan olivate | |
| D | Sodium stearoyl glutamate | 0.1 |
| D | C10-18 Triglycerides | 3 |
| D | Cetearyl alcohol | 2 |
| D | Dimethyl isosorbide | 2 |
| D | Octyldodecyl myristate | 3 |
| D | Butyrospermum parkii butter | 2 |
| D | Coco caprylate | 3 |
| E | Tromethamine A 20% | QS pH 6.5 |
| F | Argan oil | 2 |
| F | Marula oil | 2 |
| G | 1% hyaluronate gel | 2 |
| H | Albizia julibrissin bark extract | 0.5 |
| H | Tripeptide-9 citrulline | 2 |
| I | Osmosed water | 3 |
| I | Punica granatum flower | 1 |
| J | Saccharide isomerate | 2 |
| K | Fragrance | 1 |
| K | Tocopheryl acetate | 0.1 |

In a tank, phase D is heated to 70° -75° C. in a water bath. It is verified that it is thoroughly homogenous.

The amounts of osmosed water of phase I are sampled. The amount of osmosed water is introduced into the production tank, which is an open tank, at 70° -75° C., and the various ingredients of phase A are dispersed therein. Stirring is carried out with a turbine until complete dissolution is obtained. Then, with the tank open, phase B is incorporated while thoroughly wetting the powder with a spatula. Stirring is carried out with a turbine until complete homogenization is obtained. With the tank open, phase C is then incorporated while thoroughly wetting the powder with a spatula. Stirring is carried out with a turbine and planetary stirrer. With the tank open, phase D is added to the production tank. Stirring is carried out with a turbine and planetary stirrer for 10 minutes. The cooling is then begun with stirring with a turbine and planetary stirrer.

Preparation of phase I:

The various ingredients of phase I are added to the amount of cooled osmosed water indicated, with turbine stirring.

Stirring is carried out until a homogeneous smooth phase is obtained. Phase E is added, at 50° C., with turbine stirring and then stirring with a planetary stirrer for 10 minutes. At 35° C., the ingredients of phase F are successively added with turbine stirring then stirring with a planetary mixer. Phase G is added with turbine stirring and then stirring with a planetary stirrer. The ingredients of phase H are successively added with turbine stirring and then stirring with a planetary stirrer. Phase I is incorporated with turbine stirring and then the planetary stirrer is turned on. Phase J is added with turbine stirring and then the planetary stirrer is turned on. The ingredients of phase K are added successively with turbine stirring then stirring with a planetary stirrer. The turbine stirring and stirring with a planetary stirrer are continued until cooling to 25° C. is obtained.

### EXAMPLE 4

### Serum

| Phase | Ingredients | % |
|---|---|---|
| A | Osmosed water | QS 100 |
| A | Preservative | QS |
| A | Glycerin | 3 |
| A | Propanediol | 4 |
| B | Xanthan gum | 0.2 |
| C | Sodium acryloyldimethyl- | 0.2 |
| | taurate/VP crosspolymer | |
| D | Ceteareth-30 | 0.2 |
| D | Cyclopentasiloxane and | 5 |
| | cyclohexasiloxane | |
| E | Dimethyl sosorbide | 3 |
| E | Dioscorea villosa root extract | 0.1 |
| F | Albizia julibrissin bark extract | 4 |
| F | Tripeptide-9 citrulline | 2 |
| G | 1% Hyaluronate gel | 5 |
| H | Osmosed water | 5 |
| H | Punica granatum flower | 1 |
| I | Saccharide isomerate | 1 |
| J | Tocopheryl acetate | 0.1 |
| J | Fragrance | 0.3 |
| K | Tromethamine at 20% | QS pH 5.5 |

In a tank, phase D is heated to 70° -75° C. in a water bath. It is verified that it is thoroughly homogeneous. The amount of osmosed water of phase H is sampled. The amount of osmosed water is introduced into the production tank, which is an open tank, at 70° -75° C., and the various ingredients of phase A are dispersed. Stirring is carried out with the turbine until complete dissolution. With the tank open, incorporate phase B into phase A while thoroughly wetting the powder with a spatula. Stirring is carried out with a turbine until complete homogenization is obtained. With the tank open, phase C is incorporated while thoroughly wetting the powder with a spatula. Stirring is carried out with a turbine. With the tank open, phase D is added to the production tank. Standard turbine and planetary stirring is carried out for 10 minutes. The cooling is begun with turbine and planetary stirring.

Preparation of phase H:

The various ingredients of phase H are added to the amount of cooled osmosed water indicated, with stirring. Stirring is carried out until a homogeneous smooth phase is obtained. Phase E is added, at 50° C., with turbine stirring then stirring with a planetary stirrer for 10 minutes. At 35° C., the ingredients of phase F are successively added with turbine stirring and then stirring with a planetary stirrer.

Still with turbine stirring and then stirring with a planetary stirrer, phase G, then phase H and phase I and, finally, successively the ingredients of phase J, and phase K, are added. The turbine stirring and stirring with a planetary stirrer are continued until a temperature of 25° C. is reached.

1-13. (canceled)

14. A composition comprising at least one silk tree extract, one pomegranate tree extract, and one peptide of sequence SEQ ID NO: 1 in a physiologically acceptable medium.

15. The composition of claim 14, wherein the silk tree extract is a silk tree bark extract.

16. The composition of claim 14, wherein the silk tree extract is an aqueous-alcoholic extract.

17. The composition of claim 14, wherein the pomegranate tree extract is a pomegranate tree flower extract.

18. The composition of claim 17, wherein the pomegranate tree flower extract is an aqueous-alcoholic extract.

19. The composition of claim 14, wherein the final concentration of dry silk tree extract is from 0.1% to 10% by weight of the total composition.

20. The composition of claim 14, wherein the final concentration of dry pomegranate tree flower extract is from 0.1% to 10% by weight of the total composition.

21. The composition of claim 14, wherein the concentration of peptide of sequence SEQ ID NO: 1 is from 0.01% to 10% by weight of the total composition.

22. The composition of claim 14, further comprising an aqueous phase.

23. The composition of claim 14, further comprising water in a content ranging from 1% to 99% by weight relative to the total weight of the composition.

24. The composition of claim 23, further comprising water in a content ranging from 40% to 75% by weight relative to the total weight of the composition.

25. The composition of claim 14, wherein the composition is an oil-in-water emulsion, a gel or a cream, or a stick.

26. A method for combating skin aging and/or withering comprising topically applying the composition of claim 14 to the skin.
